(19) United States
(12) Patent Application Publication
(10) Pub. No.: US 2008/0177251 A1
(43) Pub. Date: Jul. 24, 2008

(54) Suction Structure Used For Surgial Operation

(76) Inventor: George Lee, Taipei (TW)

Correspondence Address: HDSL, 4331 STEVENS BATTLE LANE, FAIRFAX, VA, 22033, US

(21) Appl. No.: 11/625,390

(22) Filed: Jan. 22, 2007

(51) Int. Cl. A61M 1/00

(52) U.S. Cl. 604/540

(57) ABSTRACT

A suction device for surgical operation is used for the suction of a human nervous system and includes a flow controller and suction tube. The suction tube is a metal tube having an end connected to the flow controller and the other end forming a taper portion, and the taper portion includes a suction inlet, an externally curved inverting corner formed at an external side of the suction inlet, and an internally curved inverting corner formed at an internal side of the suction inlet, such that the suction device can prevent injuries to human tissues during the suction process and avoid blood clots from clogging the suction tube, so as to enhance the safety of use and the smoothness of the suction.

### BACKGROUND OF THE INVENTION

1. Field of the Invention

The present invention relates to a suction structure, and more particular to a suction structure used for a human nervous system.

2. Description of Prior Art

During a surgical operation of any part of a human body, it is common to suck fluids including blood or body fluid by a suction device. However, the manufacture of suction tubes and flow controllers of the present suction devices still have drawbacks that require improvements, and thus the inventor of the present invention intends to overcome the shortcomings of the prior art by providing a suction device for surgical operation.

Referring to FIGS. 1 and 2 for a conventional suction structure for surgical operation, a suction tube as shown in FIG. 1 includes a tube 20a and a fixing ring 21a. The tube 20a is provided for a surgical doctor to adjust the direction and position for bending the suction structure. The tube 20a is generally made of a metal, and a plastic fixing ring 21a is sheathed onto a suction inlet of the tube 20a. The price of such suction tube is relatively high, since both of the aforementioned material cost and manufacturing cost are incurred. In FIG. 2, an external oblique inverting corner is formed at an external side of a suction inlet of a suction tube 20b, and the shape of the external oblique inverting corner is a sharp corner, and thus it may scrape or injure human tissues easily during a suction process.

In FIG. 3, an externally curved inverting corner is formed at an external side of the suction inlet of the suction tube 20. Although such arrangement can solve part of the aforementioned problem, the surface of the internal periphery of the suction inlet is still rough and sharp. Furthermore, the internal diameter of the suction inlet of the foregoing embodiment is the same as that internal diameter of each tube 20a, 20b, 20c, and thus a larger blood clot may clog the tube 20a, 20b, 20c when air is sucked from the suction inlet, and a poor suction effect or a failure of suction will result. Therefore, it is necessary to change the suction tube or rinse the suction tube from time to time, and that makes the use of the suction device very troublesome and inconvenient, and such prior art definitely requires improvements.

### SUMMARY OF THE INVENTION

In view of the foregoing shortcomings of the prior art, the inventor of the present invention based on years of experience in the related industry to conduct experiments and modifications, and finally invented a suction structure used for surgical operation.

It is a primary objective of the present invention to provide a suction structure used for surgical operation, and the structure adopts a taper portion of the suction tube to prevent injuries to human tissues during the suction process, and also prevent blood clots from clogging the suction tube, so as to enhance the safety of use and the smoothness of the suction.

Another objective of the present invention is to provide a suction structure used for surgical operation, and the structure has an air control portion of the flow controller to facilitate controlling and adjusting the level of suction of the suction tube.

To achieve the foregoing objectives, the present invention provides a suction structure used for surgical operation, and the structure is applied for the suction of a human nervous system, and the structure includes a flow controller and a suction tube, wherein the suction tube is a metal tube having an end connected to the flow controller and a tube opening at another end forming a taper portion, and the taper portion includes a suction inlet, an externally curved inverting corner formed at an external side of the suction inlet and an internally curved inverting corner formed at an internal side of the suction inlet.

### BRIEF DESCRIPTION OF DRAWINGS

The features of the invention believed to be novel are set forth with particularity in the appended claims. The invention itself however may be best understood by reference to the following detailed description of the invention, which describes certain exemplary embodiments of the invention, taken in conjunction with the accompanying drawings in which:

FIG. 1 is a cross-sectional view of a conventional suction tube;

FIG. 2 is a cross-sectional view of another conventional suction tube;

FIG. 3 is a cross-sectional view of a further conventional suction tube;

FIG. 4 is an exploded view of a flow controller and a suction tube of the present invention;

FIG. 5 is a perspective view of a suction tube of the present invention;

FIG. 6 is a cross-sectional view of Section 6-6 as depicted in FIG. 5; and

FIG. 7 is a schematic view of an application of the present invention.

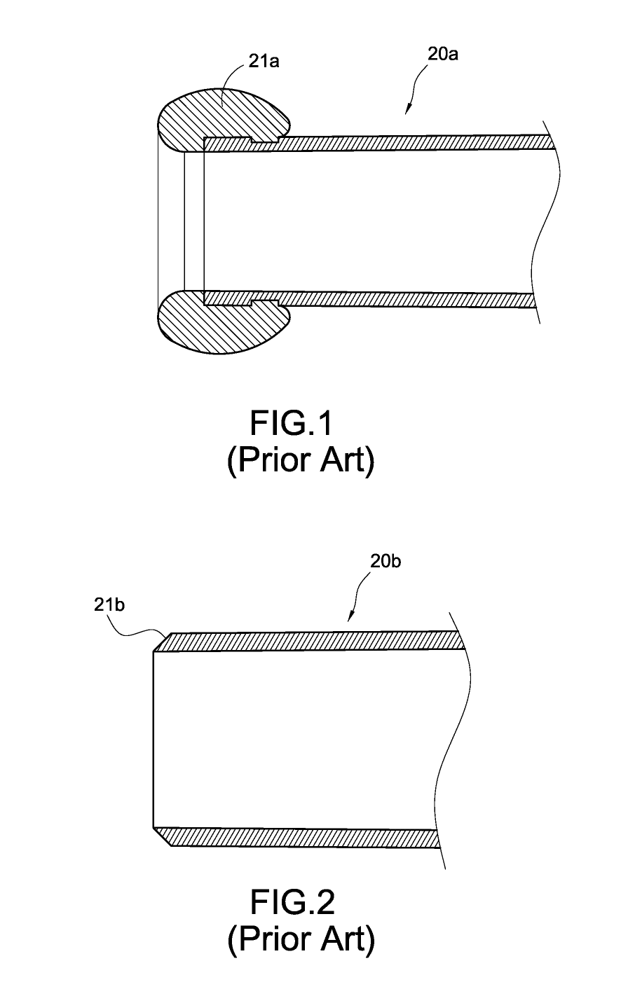

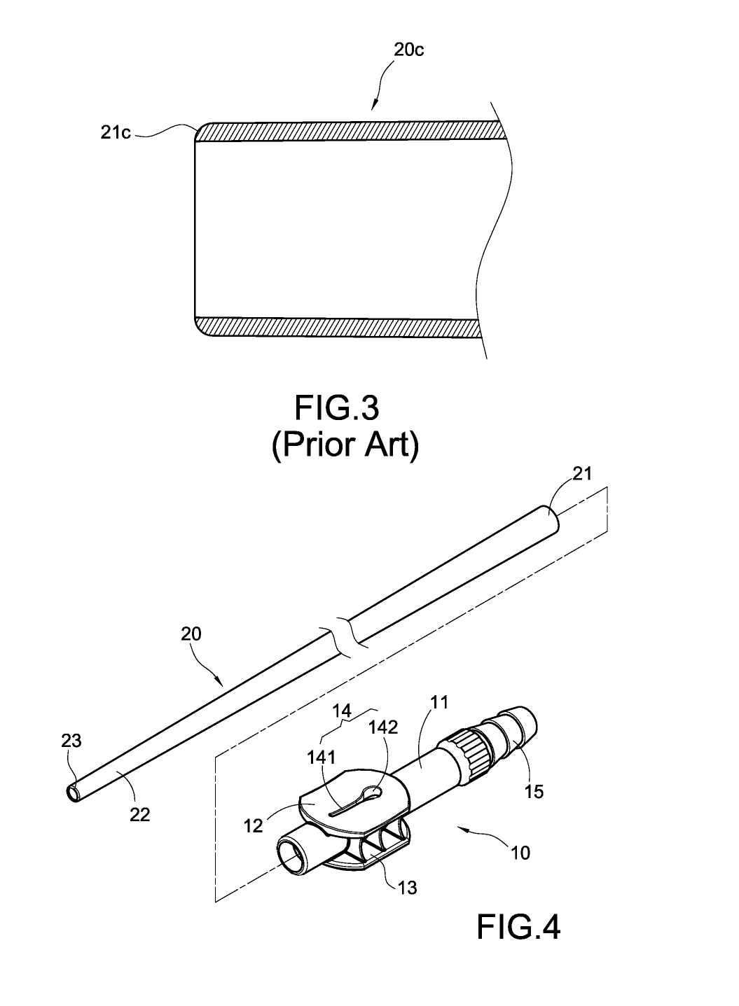

### DETAILED DESCRIPTION OF THE INVENTION

The technical characteristics, features and advantages of the present invention will become apparent in the following detailed description of the preferred embodiments with reference to the accompanying drawings. The drawings are provided for reference and illustration only, but not intended for limiting the present invention.

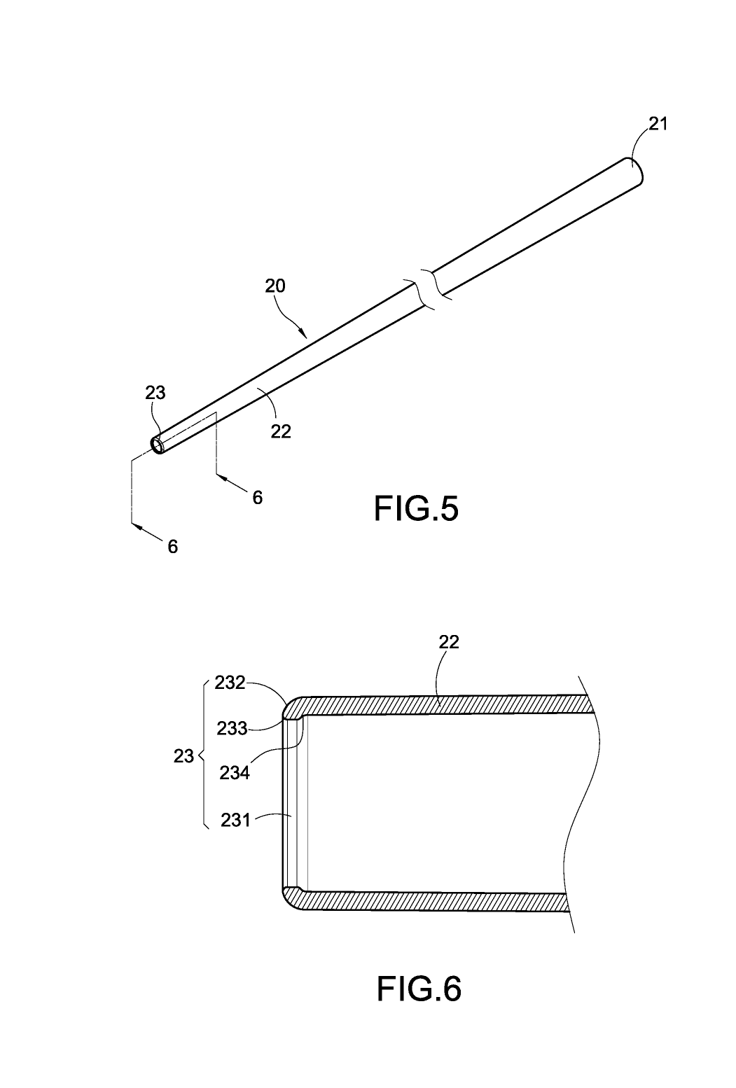

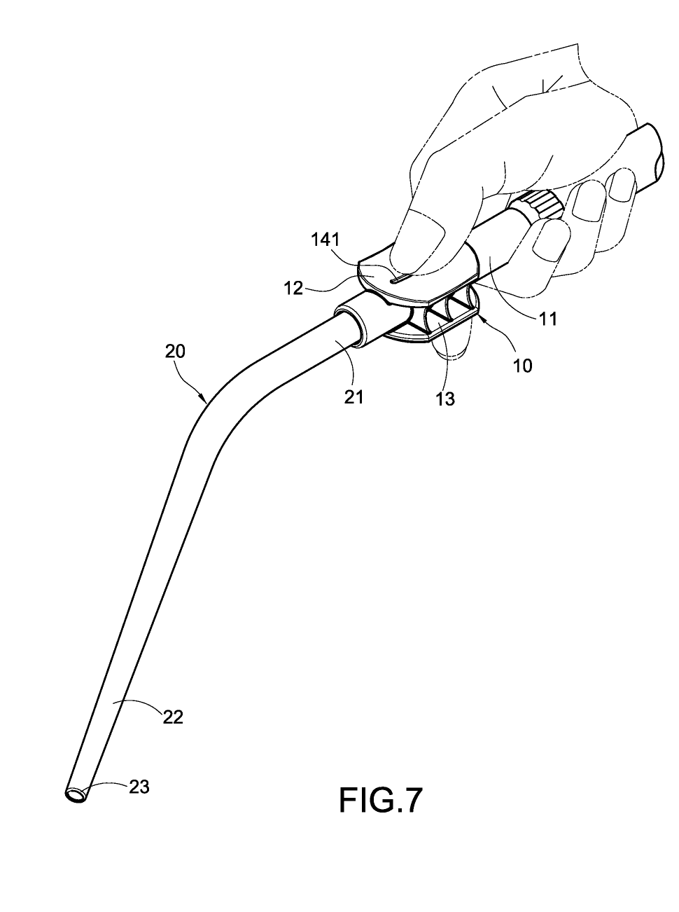

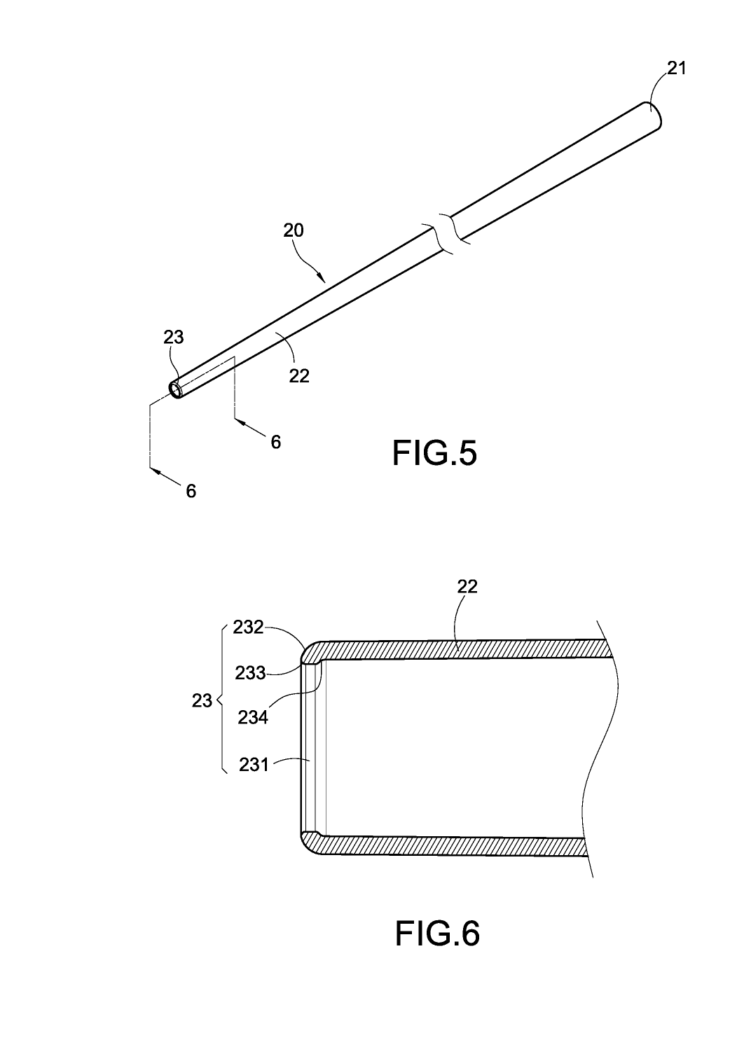

Referring to FIGS. 4 to 6 for an exploded view of a flow controller and a suction tube, a perspective view of a suction tube and a cross-sectional view of Section 6-6 as depicted in FIG. 5 in accordance with the present invention respectively, a suction structure used for the suction of a human nervous system in a surgical operation in accordance with the present invention comprises a flow controller 10 and a suction tube 20.

The flow controller 10 comprises a straight tube 11, upper and lower plates 12, 13 extended from the middle section of the straight tube 11, an air control portion 14 disposed at the middle of the upper plate 12 and penetrating inside the straight tube 11, wherein the air control portion 14 is comprised of a straight slot 141 and an arc hole 142 interconnected to an end of the straight slot 141, and a quick connector 15 extended from an end of the straight tube 11.

The suction tube 20 can be a metal tube made of aluminum, copper or stainless steel with a flexibility. In this embodiment, both ends of the suction tube 20 are made of conical tubes of two different diameters, wherein a large end 21 is inserted into the straight tube 11 of the flow controller 10, and the tube opening at a small end 22 forms a taper portion 23 by a high-speed vibration method. The taper portion 23 includes a suction inlet 231, an externally curved inverting corner 232 formed at an external side of the suction inlet 231 and an internally curved inverting corner 233 formed at an internal side of the suction inlet 231, and the internal inverting corner 233 is continued from the internal side of the external inverting corner 232 and extended into the tube, so as to prevent human tissues from being injured during the suction process and enhance the safety of use. In addition, the internal diameter of the suction inlet 231 is smaller than the internal diameter at any position of the suction tube 20, such that after a larger blood clot is sucked into the suction inlet 231, the blood clot still can flow quickly through the suction tube 20 and the interior of the flow controller 10, so as to effectively prevent blood clots from clogging the suction device and improve the smoothness of suction of the suction structure.

Referring to FIG. 7 for an application of the present invention, a negative pressure generator (not shown in the figure) is installed at the rear of a quick connector 15 of the flow controller 10, and both ends of a duct (not shown in the figure) are connected separately to the negative pressure generator and the quick connector 15, such that when the suction device is used, the negative pressure generator will be operated, and a user's thumb can seal the air control portion 14 for performing a suction of a patient's blood or blood liquid. If the concentration of the fluid in the suction structure is too high, then the user can slide the user's thumb away from the straight slot 141 to allow external air to enter into the flow controller 10 from the straight slot 141, such that the fluid can be flowed faster for a quick suction.

The present invention is illustrated with reference to the preferred embodiment and is not intended to limit the patent scope of the present invention. Various substitutions and modifications have suggested in the foregoing description, and other will occur to those of ordinary skill in the art. Therefore, all such substitutions and modifications are intended to be embraced within the scope of the invention as defined in the appended claims.

1. A suction structure used for surgical operation, comprising:
a flow controller; and
a suction tube, being a metal tube, with an end connected to the flow controller and another end forming a taper portion, and the taper portion including a suction inlet, an externally curved inverting corner formed at an external side of the suction inlet an internally curved inverting corner formed at the external side of the suction inlet and an internally curved corner formed at an internal side of the suction inlet.

2. The suction structure used for surgical operation of claim 1, wherein the flow controller comprises a straight tube, upper and lower plates extended separately from the middle of the straight tube, and an air control portion extended from the middle of the upper plate and penetrating the interior of the straight tube.

3. The suction structure used for surgical operation of claim 2, wherein the air control portion is comprised of a straight slot and an arc hole interconnected to an end of the straight slot.

4. The suction structure used for surgical operation of claim 2, wherein the straight tube has a quick connector extended from an end of the straight tube.

5. The suction structure used for surgical operation of claim 1, wherein the suction tube is a flexible tube.

6. The suction structure used for surgical operation of claim 5, wherein the suction tube is a conical tube.

7. The suction structure used for surgical operation of claim 6, wherein the suction tube is an aluminum tube.

8. The suction structure used for surgical operation of claim 6, wherein the suction tube is a copper tube.

9. The suction structure used for surgical operation of claim 6, wherein the suction tube is a stainless steel tube.

10. The suction structure used for surgical operation of claim 1, wherein the suction inlet of the taper portion has an internal diameter smaller than the internal diameter at any position of the suction tube.
